(19) United States
(12) Patent Application Publication
(10) Pub. No.: US 2015/0359750 A1
(43) Pub. Date: Dec. 17, 2015

(54) POSITIONING IMMEDIATE-RELEASE BIOADHESIVE AND APPLICATION THEREOF

(71) Applicant: Ping WAN, Shanghai (CN)

(72) Inventor: Ping WAN, Shanghai (CN)

(21) Appl. No.: 14/764,046

(22) PCT Filed: Jan. 23, 2014
(86) PCT No.: PCT/CN2014/071294

§ 371 Date: Jul. 28, 2015

(30) Foreign Application Priority Data
Jan. 28, 2013 (CN) 201310029525.3

(51) Int. Cl. A61K 9/50; A61K 9/48; A61K 9/20; A61K 9/28

(52) U.S. Cl. CPC A61K 9/5047; A61K 9/28; A61K 9/4891; A61K 9/2077

(57) ABSTRACT

Provided is a positioning immediate-release bioadhesive and relates to a medical device for preventing or/and treating diabetes, obesity, alcoholism, gastric and intestinal mucosal inflammation or/and ulcers and the like. The positioning immediate-release bioadhesive is prepared by preparing a biocompatible bioadhesive material into micro-particles, externally adding an immediate-release disintegrant, tabletting and then performing enteric or gastric coating; or performing enteric or gastric coating on the micro-particles; or filling into enteric-coated or gastric-coated hollow capsules; or directly tabletting the bioadhesive material, the immediate-release disintegrant and other additional agents and then performing enteric or gastric coating. After the positioning immediate-release bioadhesive is taken, the positioning immediate-release bioadhesive can realize positioning and immediate release, rapidly adhere to and cover the upper segment of duodenum and jejunum or/and gastric mucosa and weaken the absorption of the upper segment of duodenum and jejunum or/and stomach. The positioning immediate-release bioadhesive is orally administered, convenient to carry, convenient to store and convenient to use, and a patient using the positioning immediate-release bioadhesive does not need to go to a hospital, perform an operation or use endoscopy and further has no pain, thereby enhancing the compliance of a user and avoiding the complexity of the operation.

### FIELD OF THE INVENTION

This invention relates to a biocompatible medical device for internal use or/and external use, in particular to a positioning immediate-release bioadhesive for preventing or/and treating diabetes, obesity, alcoholism, gastric and intestinal mucosal inflammation or/and ulcers and the like.

### BACKGROUND OF THE INVENTION

In March 2011, on the Second International Congress of Interventional Treatment of Type 2 Diabetes held in New York, America, the International Diabetes Federation (IDF) firstly declared that a stomach circulation surgery could be used for treating obese patients with type 2 diabetes and reducing occurrence and development of chronic complications of the diabetes (Chinese Medical Science, 2011, 1(22): 1-2). Such surgery can also obviously improve hypertension, obesity, dyslipidemia and other complications of the patients (Chinese Medical Science, 2011, 1(21): 3-5). But the stomach circulation surgery has clinical risks, such as death, intestinal obstruction, anastomotic leakage, pulmonary embolism, deep vein thrombosis, portal vein injuries, respiratory diseases and the like (Chinese Journal of Diabetes, 2011, 3(3): 205-208). Recently, the way of placing a duodenum inner covering film in vivo to cover mucosa of the upper segment of duodenum and jejunum to further treat diabetes and obesity tends to replace the above “stomach circulation surgery”. However, the invention patent “duodenal casing and conveyor thereof” (with the application date of Apr. 9, 2010 and the authorization publication date of Jan. 11, 2012) in the prior art, the invention patent “duodenum inner covering film prepared from degradable biocompatible material and application thereof” (with the application date of May 5, 2012 and the publication date of Aug. 8, 2012) in the prior art and the invention patent “duodenum inner covering film prepared by electrostatic spinning” (with the application date of Aug. 21, 2012 and the publication date of Nov. 21, 2012) in the prior art are all medical devices implanted into human bodies, the implantation operation depends on endoscopy, and the non-degradable materials also need to be taken out after a period of time, thereby not only affecting the compliance of users, but also increasing the complexity of the operation (in comparison with the present invention).

The basic mechanism for surgical treatment of the obesity is to limit food intake and reduce gastric and intestinal absorption, corresponding to the invention patent “tissue conveyor used in sleeve gastrectomy and related using method” (with the application date of Apr. 30, 2009 and the publication date of Apr. 13, 2011) in the prior art, the invention patent “releasable gastroplasty ring” (with the application date of Dec. 21, 2000 and the authorization publication date of Sep. 1, 2004) in the prior art, the invention patent “gastroplasty ring with single control” (with the application date of Jan. 19, 2001 and the authorization publication date of Oct. 20, 2004) in the prior art and the like, or placement of balloon or gastric band in gastric cavity (Yang Kejun, Advantages of adjustable gastric band bariatric surgery. Shanghai Medicines, 2012, 33(8): 11; Yang Shen, et al., Clinical studies of intragastric balloon therapy for obesity. Chinese Medical Science, 2011, 1(6): 23-24; Mei Liwen, et al., Efficacy and safety evaluation of intragastric balloon therapy in patients with obesity. Chinese Medical Journal, 2007, 87(6): 388-391). By adopting the basic mechanism, although the food intake per meal can be limited and the absorption of the stomach can be reduced, the shortcomings in the compliance of the users, the complexity of the operation and the risks are self-evident.

The common hangover relief idea mostly focuses on how to passively relieve or reduce the effects after drinking, corresponding to the invention patent “composition of hangover relief oral medicament and preparation process thereof” (with the application date of Dec. 20, 2010 and the publication date of Jul. 11, 2012) in the prior art, the invention patent “anti-drunk and hangover relief composition and preparation method thereof” (with the application date of May 18, 2012 and the publication date of Sep. 19, 2012) in the prior art and the invention patent “oral absorption solid hangover relief effervescent preparation” (with the application date of Jul. 12, 2010 and the publication date of Dec. 22, 2010) in the prior art and the like. Without evaluating the efficacy of these substances in hangover relief, the substances can also increase the burden on liver or/and kidney and other organs by absorption, metabolism and other in-vivo ways, and more importantly, the nodes for passively relieving hangover are basically after organisms absorb wine, and this situation has increased the burden on the related organs of the organisms.

### SUMMARY OF THE INVENTION

### Technical Problems

The invention patent “duodenal casing and conveyor thereof” (with the application date of Apr. 9, 2010 and the authorization publication date of Jan. 11, 2012) in the prior art, the invention patent “duodenum inner covering film prepared from degradable biocompatible material and application thereof” (with the application date of May 5, 2012 and the publication date of Aug. 8, 2012) in the prior art and the invention patent “duodenum inner covering film prepared by electrostatic spinning” (with the application date of Aug. 21, 2012 and the publication date of Nov. 21, 2012) in the prior art not only affect the compliance of users, but also increase the complexity of the operation (in comparison with the invention). After the positioning immediate-release bioadhesive of the invention is orally administered, a pH-sensitive coating material transfers the enteric-coated immediate-release bioadhesive (micro-particles or/and capsules or/and tablets) to the upper segment of duodenum and jejunum in a positioning manner according to the differences in intra-gastrointestinal pH value, and the enteric-coated immediate-release bioadhesive achieving the upper segment of duodenum and jejunum rapidly or/and sharply degrades the coating material in a high-pH environment. In the intestinal cavity at the upper segment of duodenum and jejunum, the adhesive material in the enteric-coated immediate-release bioadhesive is rapidly, fully and completely released, disintegrated, floated, dissolved and swollen (the positioning immediate-release bioadhesive which is pressed into the tablets is also rapidly, fully and completely released, disintegrated, floated, dissolved and swollen due to an immediate-release disintegrant and the like), further interacted with mucosal proteins or/and mucosal epithelial cells and the like to adhere thereto immediately after being in contact with the mucosa of the upper segment of duodenum and jejunum till the enteric-coated immediate-release bioadhesive is fully adhered to and covers the mucosa of the upper segment of duodenum and jejunum or/and is embedded into folds and valley cracks of the mucosa; and the ascending part of duodenum further prolongs the retention time of the adhesive material in the duodenum while reducing the reflux of contents in the jejunum and ileum. The positioning immediate-release bioadhesive is orally administered, convenient to carry, convenient to store and convenient to use, and a patient does not need to go to a hospital, perform an operation or use endoscopy and further has no pain when taking the positioning immediate-release bioadhesive, thereby enhancing the compliance of a user (patients with obesity, patients with diabetes, alcoholism preventers, people with duodenal inflammation or/and ulcers and the like) and almost zeroing the complexity of the operation. As the positioning immediate-release bioadhesive covers the mucosa of the upper segment of duodenum and jejunum, the absorption of alcohol in the mucosa of the upper segment of duodenum and jejunum can also be reduced to reduce alcoholism; and as the positioning immediate-release bioadhesive covers the mucosa of the upper segment of duodenum and jejunum, the upper segment of duodenum and jejunum can also be protected, thereby preventing or/and treating duodenal or/and jejunal inflammation or/and ulcers. The amount and period of superimposed administration is determined according to the time of gradual degradation or/and erosion or/and dissolution of the adhesive material in vivo.

According to the invention patent “tissue conveyor used in sleeve gastrectomy and related using method” (with the application date of Apr. 30, 2009 and the publication date of Apr. 13, 2011) in the prior art, the invention patent “releasable gastroplasty ring” (with the application date of Dec. 21, 2000 and the authorization publication date of Sep. 1, 2004) in the prior art, the invention patent “gastroplasty ring with single control” (with the application date of Jan. 19, 2001 and the authorization publication date of Oct. 20, 2004) and the like, or placement of balloon or gastric band in gastric cavity (Yang Kejun, Advantages of adjustable gastric band bariatric surgery. Shanghai Medicines, 2012, 33(8): 11; Yang Shen, et al., Clinical studies of intragastric balloon therapy for obesity. Chinese Medical Science, 2011, 1(6): 23-24; Mei Liwen, et al., Efficacy and safety evaluation of intragastric balloon therapy in patients with obesity. Chinese Medical Journal, 2007, 87(6): 388-391), although the food intake per meal can be limited and the absorption of the stomach can be reduced, the shortcomings in the compliance of the users, the complexity of the operation and the risks are self-evident. By using the positioning immediate-release bioadhesive of the invention, the user (patients with obesity, patients with diabetes and other people) does not need to go to the hospital, perform the operation or use endoscopy and further has no pain, the positioning immediate-release bioadhesive only needs to be orally taken, after the administration, the pH-sensitive coating material transfers the gastric-coated immediate-release bioadhesive (micro-particles or/and capsules or/and tablets) to the stomach in a positioning manner according to the differences in the intra-gastrointestinal pH value, and the gastric-coated immediate-release bioadhesive achieving the stomach rapidly or/and sharply degrades the coating material in a gastric pH environment. In the gastric cavity, the adhesive material in the gastric-coated immediate-release bioadhesive is rapidly, fully and completely released, disintegrated, floated, dissolved and swollen (the positioning immediate-release bioadhesive which is pressed into the tablets is also rapidly, fully and completely released, disintegrated, floated, dissolved and swollen due to the immediate-release disintegrant), further interacted with mucosal proteins or/and mucosal epithelial cells and the like to adhere thereto immediately after being in contact with gastric mucosa till the gastric-coated immediate-release bioadhesive is fully adhered to and covers the gastric mucosa or/and is embedded into folds and valley cracks of the mucosa; and the pylorus of the stomach further prolongs the retention time of the adhesive material in the stomach while reducing the reflux of the contents in the duodenum. Thus, the adhesive material is adhered to and covers on the gastric mucosa and the absorption of the stomach can be reduced. The amount and period of superimposed administration is determined according to the time of gradual degradation or/and erosion or/and dissolution of the adhesive material in vivo.

As for the invention patent “composition of hangover relief oral medicament and preparation process thereof” (with the application date of Dec. 20, 2010 and the publication date of Jul. 11, 2012) in the prior art, the invention patent “anti-drunk and hangover relief composition and preparation method thereof” (with the application date of May 18, 2012 and the publication date of Sep. 19, 2012) in the prior art and the invention patent “oral absorption solid hangover relief effervescent preparation” (with the application date of Jul. 12, 2010 and the publication date of Dec. 22, 2010) in the prior art and the like, the nodes for relieving hangover are basically after organisms absorb wine, this situation has increased the burden on the related organs of the organisms, and the substances for relieving the hangover can also increase the burden on liver or/and kidney and other organs by absorption, metabolism and other in-vivo ways. Normally, after the intake of alcohol, about 80% of the alcohol is absorbed by the mucosa of duodenum and jejunum, and the remaining part is absorbed by the gastric mucosa (“Internal Medicine. Volume 1”: p 789). After the positioning immediate-release bioadhesive of the invention is orally taken, the pH-sensitive coating material transfers the gastric-coated or/and enteric-coated immediate-release bioadhesive (micro-particles or/and capsules or/and tablets) to the stomach or/and the duodenum and jejunum in a positioning manner according to the differences in the intra-gastrointestinal pH value, and the gastric-coated or/and enteric-coated immediate-release bioadhesive achieving the stomach or/and the duodenum and jejunum rapidly or/and sharply degrades the coating material in a corresponding pH environment. In the gastric cavity or/and the duodenum and jejunum cavity, the adhesive material in the gastric-coated or/and enteric-coated immediate-release bioadhesive is rapidly, fully and completely released, disintegrated, floated, dissolved and swollen (the positioning immediate-release bioadhesive which is pressed into the tablets is also rapidly, fully and completely released, disintegrated, floated, dissolved and swollen due to the immediate-release disintegrant), further interacted with mucosal proteins or/and mucosal epithelial cells and the like to adhere thereto immediately after being in contact with the mucosa of the stomach or/and the duodenum and jejunum till the gastric-coated or/and enteric-coated immediate-release bioadhesive is fully adhered to and covers the mucosa of the stomach or/and the duodenum and jejunum or/and is embedded into folds and valley cracks of the mucosa. Thus, the gastric-coated or/and enteric-coated immediate-release bioadhesive is just adhered to and covers the mucosa of the stomach or/and the duodenum and jejunum and not absorbed, thereby actively inhibiting the absorption of the wine by the stomach or/and the duodenum and jejunum; and as the gastric-coated or/and enteric-coated immediate-release bioadhesive covers the mucosa of the stomach or/and the duodenum and jejunum, the mucosa of the stomach or/and the duodenum and jejunum can also be protected, thereby preventing or/and treating inflammation or/and ulcers of the stomach or/and the duodenum and jejunum. The amount and period of superimposed administration is determined according to the time of gradual degradation or/and erosion or/and dissolution of the adhesive material in vivo.

### Technique Solutions

A positioning immediate-release bioadhesive is characterized in that the positioning immediate-release bioadhesive is prepared by preparing a biocompatible bioadhesive material into micro-particles, externally adding an immediate-release disintegrant, tabletting and then performing enteric coating; or performing enteric coating on the micro-particles; or filling into enteric-coated hollow capsules; or directly tabletting the bioadhesive material, the immediate-release disintegrant and other additional agents and then performing enteric coating; and the positioning immediate-release bioadhesive can adhere to and cover mucosa of the upper segment of duodenum and jejunum after administration and can prevent or/and treat diabetes and obesity, weaken alcohol absorption and prevent or/and treat duodenal inflammation or/and ulcers.

A positioning immediate-release bioadhesive is characterized in that the positioning immediate-release bioadhesive is prepared by preparing a biocompatible bioadhesive material into micro-particles, externally adding an immediate-release disintegrant, tabletting and then performing gastric coating; or performing gastric coating on the micro-particles; or filling into gastric-coated hollow capsules; or directly tabletting the bioadhesive material, the immediate-release disintegrant and other additional agents and then performing gastric coating; and the positioning immediate-release bioadhesive can adhere to and cover gastric mucosa, weaken alcohol absorption in the stomach, prevent or/and treat obesity and prevent or/and treat gastritis or/and ulcers.

The enteric-coated positioning immediate-release bioadhesive can be obtained by the following steps and ways:

The preparation of micro-particles is as follows:

Taking 1-5 g of lactide-polyethylene glycol copolymer (PELA) with the weight ratio of lactide to polyethylene glycol of (80-90):(20-10) and the molecular weight of the polyethylene glycol of 6000, dissolved in 15-25 ml of anhydrous ethanol, taking this as an internal phase; taking 100 ml of liquid paraffin with 2% of span 85 as an external phase; slowly dropping the internal phase into the external phase while magnetically stirring at a high speed, performing decompression at 60° C. to remove ethanol, immediately placing into an ice bath to cool to form a solid; centrifugally separating the liquid paraffin, precipitating, washing with petroleum ether and performing vacuum drying; and screening by a 100-mesh screen rather than a 200-mesh screen; and observing the shape under an optical microscope.

Or, configuring type A gelatin with the molecular weight of 50000 into a 3-8% solution, adding an agglomerant, namely sodium sulfate while stirring at the temperature of 45° C., standing, separating, washing with cold isopropanol, then cross-linking and curing with an isopropanol solution with 5-15% of formaldehyde, dehydrating, and performing vacuum drying to obtain the micro-particles, wherein water can be used as a diluent; and repeatedly performing agglomeration and deagglomeration, observing the shape under the optical microscope till the formation of the appropriate shape and further cross-linking and curing.

Or, adding water into a mixed solution with 5-15% of gelatin, 5-15% of gum arabic and 70-90% of water for gradual dilution, observing the shape under the optical microscope till the formation of an appropriate shape and further cross-linking and curing.

Or, dissolving a ternary system consisting of polyisobutylene, ethyl cellulose and cyclohexane at 80° C. to form a uniform solution, slowly cooling to 45° C. and further rapidly cooling to 25° C. to form the micro-particles.

Or, (preparing nano-particles), placing a 300 g/L gelatin solution into an equal amount of sesame oil for emulsification, placing an emulsion into the ice bath to gelatinize gelatin emulsion droplets, diluting with acetone, filtering with a 50 nm filter membrane, rinsing oil on nanospheres with the acetone, cross-linking with an acetone solution with 5-15% of formaldehyde for 5-15 min and drying for preparation.

Or, (preparing the nano-particles), ultrasonically dissolving 100 mg of PLGA in 5-15 ml of acetone, dropping into 30-50 ml of water solution with 0.01-0.05% of carbomer while magnetically stirring, stirring at room temperature at 500 rpm till an organic solvent evaporates completely, centrifugating at 4° C. and 15000 rpm for 20-40 min, discarding supernatant, removing a residual surfactant, re-dissolving a precipitate in Millipore water, washing with water for three times and drying to obtain the PLGA nano-particles.

Or, (preparing the nano-particles), dissolving chitosan in a dilute acetic acid water solution, swelling overnight, preparing into a 0.3-1.0% (w/v) chitosan solution, dissolving sodium tripolyphosphate in distilled water to prepare a 0.3-1.0% (w/v) solution, continuously performing magnetic stirring, and adding a sodium tripolyphosphate solution into the chitosan solution at the dropping speed of about 2-5 ml/min, wherein the solution gradually turns from clear to light blue opalescent, and the formation of the nano-particles is judged according to opalescence.

Or, (preparing the nano-particles), dissolving the PLGA in trifluoroethanol at normal temperature for 36-72 hr, performing magnetic stirring till 5-50% w/v, transferring the solution into a micro-infusion pump connected with a high-pressure generator, regulating the voltage V to 5-35 kV, the receiving distance L to 1-20 cm and the flow rate f of the solution to 0.1-2.0 ml/h, performing electrospraying, receiving the obtained micro-particles by an aluminum foil receiving plate or a glass slide, and drying in a drying box for 2 d to obtain the nano-particles; and observing the appearance of the prepared micro-particles under a scanning electron microscope.

The direct tabletting is performed as follows:

Uniformly mixing 1 part of carbomer 934P and 1 part of carboxymethylcellulose sodium 2000 cp and directly tabletting powder till the thickness is 1-3 mm, the diameter is 3-13 mm and the hardness is about 4 kg/mm²; or performing wet process granulation and tabletting, wherein a binder can select a 60-80% ethanol solution with 3-10% of PVP_{K30}, a lubricant can select magnesium stearate (1-5%) and a filler can select pregelatinized starch.

Or, screening 30-50% of mannitol, 30-40% of microcrystalline cellulose and an appropriate amount of lactose by a 100-mesh screen, performing equal-amount gradual increase and uniform mixing, adding a 5% polyvinylpyrrolidone K30 solution as the binder, granulating, drying at 60° C. for 0.5-2 h, performing size stabilization, and further uniformly mixing an appropriate amount of carboxymethylcellulose sodium and micro-powder silica gel and tabletting.

Or, screening sodium bicarbonate and magnesium hydroxide in the ratio of 1:2, 0.5-2% of magnesium stearate, 1-5% of cross-linked carboxymethylcellulose sodium and 5-15% of Starch 1500 by a 100-mesh screen, totally and uniformly mixing, and tabletting till the hardness is 4-10 kg/mm².

The tabletting of the micro-particles can be performed as follows:

Screening raw materials and auxiliary materials by the 100-mesh screen, uniformly mixing, adding the binder, namely a 3-15% PVP water solution, to prepare a soft material, granulating, and drying at 60° C. for 0.5-2 h; and adding the magnesium stearate or/and the diluent or/and the wetter and the like, performing size stabilization and tabletting for preparation.

The enteric coating is performed as follows:

Taking hydroxypropyl methylcellulose phthalate with a pH sensitive point of 5-6 and preparing into a 1.0-3.0% solution with acetone/ethanol (1/1, v/v), wherein the using amount of the additional agents is 10-30%; uniformly mixing and regulating the coating weight gain to 1-5%; regulating the rotational speed of a coating pan to enable tablet cores to roll, rotate and be polished in a parabolic manner for about 60±5 r/min; preheating the tablet cores by air which is blown in by an air blower, regulating the air inlet position and the air outlet speed at the temperature of about 50° C. to uniformly spray out a coating solution; observing the tablet cores after 10-30 min to find that the edges are smooth and have no defects or splits, the coated tablets can prevent sticking and coating films are uniform and smooth; taking out the tablet cores after coating, and drying in an oven at the temperature of about 60° C.; and weighing and taking the coating weight gain by percentage as a coating control index.

Or, taking 3-5% of EC, 0.3-1.0% of DEP and 0.1-0.6% of PEG 400 and taking a 60-90% ethanol water solution as a coating solvent; placing the tablet cores into the coating pan and preheating, wherein the inclination angle of the coating pan is 45° and the inner diameter of a spray nozzle is 0.5-1.0 mm; the atomization pressure of a spray gun is about 137.3 kPa, the air inlet temperature is 35±5° C. and the tablet temperature is 35±2° C.; and the rotational speed is 13-36 r/min and the spraying speed is 0.5-1.0 ml/min.

Or, immersing the tablet cores into a 1-5% (WN) Eudragit L100-55 acetone solution, taking out after 2-10 min and drying, repeating for 3-6 times and controlling the thickness at about 50 μm.

The enteric coating of the micro-particles can be performed as follows:

Placing the prepared micro-particles into a fluidized bed coating device for boiling and fluidizing, spraying an ethanol solution with 4-8% of acrylic resin by the spray gun, blasting, drying, and exhausting and evaporating the solvent from an exhaust port to obtain the enteric-coated micro-particles with uniform coating thickness and no adhesion.

The filling of enteric-coated capsules and the micro-particles can be performed as follows:

Using a high-efficiency coating machine, wherein the diameter of the spray nozzle is 0.5-1.5 mm, the atomization pressure is 0.1 MPa, the air volume is 50-120 m³/h, the material temperature is 23-25° C. and the spraying speed is 0.5-5.5 g/min; determining the thickness by a digital display micrometer, curing at 25° C. for 20-60 min, taking out the enteric-coated capsules after coating and drying at room temperature; filling the enteric-coated hollow capsules, sealing with a 5-15% ethyl cellulose solution and placing into a dryer for later use, wherein an appropriate amount of anti-sticking agent, namely magnesium stearate or silicon dioxide and the like, or the diluent, the lubricant, the disintegrant and the like can be added.

The enteric coating material:

can be Eudragit L type, Eudragit S type, acrylic resin No. I, acrylic resin No. II, acrylic resin No. III, acrylic resin No. IV, cellulose acetate phthalate (CAP), 1,2,4-cellulose acetate trimellitate (CAT), cellulose acetate succinate (CAS), hydroxypropylmethylcellulose phthalate (HPMCP), 1,2,4-hydroxypropylmethylcellulose trimellitate (HPMCT), hydroxypropylmethylcellulose acetate succinate (HPMCAS) and other materials.

The gastric-coated positioning immediate-release bioadhesive can be obtained by the following steps:

The preparation of micro-particles is as follows:

Taking 1-2 parts of solution formed by dissolving 5-25% of ethyl cellulose-carbomer 934P copolymer in anhydrous ethanol, stirring in a water bath at the temperature of 5-15° C. for 20-30 min, slowly dropping into 5-7 parts of liquid paraffin with 1-10% of span 85 at the temperature of 5-15° C. at a uniform speed, stirring for 30-40 min, performing decompression at 60° C. to remove ethanol, immediately placing into an ice bath to cool to form a solid, centrifugally separating the liquid paraffin, precipitating, washing with petroleum ether and drying in a drying box at 37° C. for 12-24 hours, screening by a 100-mesh screen rather than a 200-mesh screen; and observing the shape under an optical microscope.

Or, configuring type A gelatin with the molecular weight of 50000 into a 3-8% solution, adding an agglomerant, namely sodium sulfate while stirring at the temperature of 45° C., standing, separating, washing with cold isopropanol, then cross-linking and curing with an isopropanol solution with 5-15% of formaldehyde, dehydrating, and performing vacuum drying to obtain the micro-particles, wherein water can be used as a diluent; and repeatedly performing agglomeration and deagglomeration, observing the shape under the optical microscope till the formation of the appropriate shape and further cross-linking and curing.

Or, adding water into a mixed solution with 5-15% of gelatin, 5-15% of gum arabic and 70-90% of water for gradual dilution, observing the shape under the optical microscope till the formation of the appropriate shape and further cross-linking and curing.

Or, dissolving a ternary system consisting of polyisobutylene, ethyl cellulose and cyclohexane at 80° C. to form a uniform solution, slowly cooling to 45° C. and further rapidly cooling to 25° C. to form the micro-particles.

Or, (preparing nano-particles), placing a 300 g/L gelatin solution into an equal amount of sesame oil for emulsification, placing an emulsion into the ice bath to gelatinize gelatin emulsion droplets, diluting with acetone, filtering with a 50 nm filter membrane, rinsing oil on nanospheres with the acetone, cross-linking with an acetone solution with 5-15% of formaldehyde for 5-15 min and drying for preparation.

Or, (preparing the nano-particles), ultrasonically dissolving 100 mg of PLGA in 5-15 ml of acetone, dropping into 30-50 ml of water solution with 0.01-0.05% of carbomer while magnetically stirring, stirring at 500 rpm at room temperature till the organic solvent evaporates completely, centrifugating at 4° C. and 15000 rpm for 20-40 min, discarding supernatant, removing a residual surfactant, re-dissolving a precipitate in Millipore water, washing with water for three times and drying to obtain the PLGA nano-particles.

Or, (preparing the nano-particles), dissolving chitosan in a dilute acetic acid water solution, swelling overnight, preparing into a 0.3-1.0% (w/v) chitosan solution, dissolving sodium tripolyphosphate in distilled water to prepare a 0.3-1.0% (w/v) solution, continuously performing magnetic stirring, and adding a sodium tripolyphosphate solution into the chitosan solution at the dropping speed of about 2-5 ml/min, wherein the solution gradually turns from clear to light blue opalescent, and the formation of the nano-particles is judged according to opalescence.

Or, (preparing the nano-particles), dissolving the PLGA in trifluoroethanol at normal temperature for 36-72 hr, performing magnetic stirring till 5-50% w/v, transferring the solution into a micro-infusion pump connected with a high-pressure generator, regulating the voltage V to 5-35 kV, the receiving distance L to 1-20 cm and the flow rate f of the solution to 0.1-2.0 ml/h, performing electrospraying, receiving the obtained micro-particles by an aluminum foil receiving plate or a glass slide, and drying in the drying box for 2 d to obtain the nano-particles; and observing the appearance of the prepared micro-particles under a scanning electron microscope.

The direct tabletting is performed as follows:

Uniformly mixing 1 part of carbomer 934P and 1 part of sodium carboxymethyl cellulose 2000 cp and directly tabletting powder till the thickness is 1-3 mm, the diameter is 3-13 mm and the hardness is about 4 kg/mm²; or performing wet process granulation and tabletting, wherein a binder can select a 60-80% ethanol solution with 3-10% of PVP_{K30}, a lubricant can select magnesium stearate (1-5%) and a filler can select pregelatinized starch.

Or, screening 30-50% of mannitol, 30-40% of microcrystalline cellulose and an appropriate amount of lactose by a 100-mesh screen, performing equal-amount gradual increase and uniform mixing, adding a 5% polyvinylpyrrolidone K30 solution as the binder, granulating, drying at 60° C. for 0.5-2 h, performing size stabilization, further uniformly mixing an appropriate amount of carboxymethylcellulose sodium and micro-powder silica gel and tabletting.

Or, screening sodium bicarbonate and magnesium hydroxide in the ratio of 1:2, 0.5-2% of magnesium stearate, 1-5% of cross-linked carboxymethylcellulose sodium and 5-15% of Starch 1500 by a 100-mesh screen, totally and uniformly mixing, and tabletting till the hardness is 4-10 kg/mm².

The tabletting of the micro-particles can be as follows:

Screening raw materials and auxiliary materials by the 100-mesh screen, uniformly mixing, adding the binder, namely a 3-15% PVP water solution, to prepare a soft material, granulating, and drying at 60° C. for 0.5-2 h; and adding the magnesium stearate or/and the diluent or/and the wetter and the like, performing size stabilization and tabletting for preparation.

The gastric coating is as follows:

Taking gastric-coated acrylic resin (No. VI) with a pH sensitive point of 1-2 and preparing into a 2.0% solution with acetone/ethanol (1/1, v/v), wherein the using amount of the additional agents is 10-50%; uniformly mixing and regulating the coating weight gain to 1-5%; regulating the rotational speed of a coating pan to enable tablet cores to roll, rotate and be polished in a parabolic manner for about 60±5 r/min; preheating the tablet cores by air which is blown in by an air blower, regulating the air inlet position and the air outlet speed at the temperature of about 50° C. to uniformly spray out a coating solution; observing the tablet cores after 10-15 min to find that the edges are smooth and have no defects or splits, the coated tablets can prevent sticking and coating films are uniform and smooth; taking out the tablet cores after coating, and drying in an oven at the temperature of about 60° C.; and weighing and taking the coating weight gain by percentage as a coating control index.

The gastric coating of the micro-particles can be as follows:

Placing the prepared micro-particles into a fluidized bed coating device for boiling and fluidizing, spraying a 5-7% ethanol hydroxypropyl methyl cellulose solution by the spray gun, blasting, drying, and exhausting and evaporating the solvent from an exhaust port to obtain the gastric-coated micro-particles with uniform coating thickness and no adhesion.

The filling of gastric-coated capsules and the micro-particles can be performed as follows:

Using a high-efficiency coating machine, wherein the diameter of the spray nozzle is 0.5-1.5 mm, the atomization pressure is 0.1 MPa, the air volume is 50-120 m³/h, the material temperature is 23-25° C. and the spraying speed is 0.5-5.5 g/min; determining the thickness by a digital display micrometer, curing at 25° C. for 20-60 min, taking out the gastric-coated capsules after coating and drying at room temperature; filling the gastric-coated hollow capsules, sealing with a 5-15% ethyl cellulose solution and placing into a dryer for later use, wherein an appropriate amount of anti-sticking agent, namely magnesium stearate or silicon dioxide and the like, or the diluent, the lubricant, the disintegrant and the like can be added.

The gastric coating material:

can be hydroxypropyl methylcellulose (HPMC), methyl cellulose (MC), polyvinyl alcohol (PVA), hydroxypropyl cellulose (HPC), polyethylene glycol (PEG), polyvinyl acetal diethylamino acetate (AEA), Eudragit E type, gastric-coated acrylic resin and other materials.

The bioadhesive material of the enteric-coated or gastric-coated positioning immediate-release bioadhesive of the invention:

can be carbomer (CP), hydroxypropyl methylcellulose (HPMC), hydroxypropyl cellulose (HPC), ethylenediamine-modified polylactic acid (EMPLA), polytetrafluoroethylene, polylactic acid-glycolic acid (PLGA), polylactic acid-caprolactone (PCL-b-LA), poly-ε-caprolactone (PCL), silicone oil, silicone rubber, polyester-polyether copolymer, grafted polylactic acid, gelatin, bletilla hyacinthine gum, alginate, cellulose derivatives, chitosan, lectin (phytohaemagglutinin), tomato lectin, N-(2-hydroxypropyl) methacrylamide copolymer and other materials. The using amount of the adhesive material is 10%-90%. The filler can be lactose, microcrystalline cellulose, sucrose, starch, pregelatinized starch and other materials. The binder can be water, ethanol with different concentrations, PVP_{K30} with different concentrations and other materials.

In the preparation of the enteric-coated or gastric-coated positioning immediate-release bioadhesive micro-particles of the invention, a solvent evaporation method, a spray drying method, a phase separation method, an electrospraying method, an acoustic excitation atomization method, an emulsion polymerization method, an interfacial polymerization method, an in-situ polymerization method, a polymer rapid insolubilization method, an atomization spray extraction method, a single-emulsion method, a double-emulsion method, an intermediate phase separation method, a drying method in a solution, a solution evaporation method, a powder bed method, an air suspension coating method, a vacuum spraying method, an electrostatic aerosol method, a porous centrifugation method and other methods can be adopted.

The amount and period of superimposed administration is determined according to the time of gradual degradation or/and erosion or/and dissolution of the adhesive material in vivo.

### The Invention has the Following Beneficial Effects

An enteric-coated positioning immediate-release bioadhesive is provided, compared with the prior art (the invention patent “duodenal casing and conveyor thereof”; the invention patent “duodenum inner covering film prepared from degradable biocompatible material and application thereof”; and the invention patent “duodenum inner covering film prepared by electrostatic spinning”), after the positioning immediate-release bioadhesive of the invention is orally taken, the pH-sensitive coating material transfers the enteric-coated immediate-release bioadhesive (micro-particles or/and capsules or/and tablets) to the upper segment of duodenum and jejunum in a positioning manner according to the differences in intra-gastrointestinal pH value, and the enteric-coated immediate-release bioadhesive achieving the upper segment of duodenum and jejunum rapidly or/and sharply degrades the coating material in a high-pH environment. In the intestinal cavity at the upper segment of duodenum and jejunum, the adhesive material in the enteric-coated immediate-release bioadhesive is rapidly, fully and completely released, disintegrated, floated, dissolved and swollen (the positioning immediate-release bioadhesive which is pressed into the tablets is also rapidly, fully and completely released, disintegrated, floated, dissolved and swollen due to an immediate-release disintegrant and the like), further interacted with mucosal proteins or/and mucosal epithelial cells and the like to adhere thereto immediately after being in contact with the mucosa of the upper segment of duodenum and jejunum till the enteric-coated immediate-release bioadhesive is fully adhered to and covers the mucosa of the upper segment of duodenum and jejunum or/and is embedded into folds and valley cracks of the mucosa; and the ascending part of duodenum further prolongs the retention time of the adhesive material in the duodenum while reducing the reflux of contents in the jejunum and ileum. The positioning immediate-release bioadhesive is orally administered, convenient to carry, convenient to store and convenient to use, and a patient does not need to go to a hospital, perform an operation or use endoscopy and further has no pain when taking the positioning immediate-release bioadhesive, thereby enhancing the compliance of a user (patients with obesity, patients with diabetes, alcoholism preventers, people with duodenal inflammation or/and ulcers and the like) and almost zeroing the complexity of the operation. As the positioning immediate-release bioadhesive covers the mucosa of the upper segment of duodenum and jejunum, the absorption of alcohol in the mucosa of the upper segment of duodenum and jejunum can also be reduced to reduce alcoholism; and as the positioning immediate-release bioadhesive covers the mucosa of the upper segment of duodenum and jejunum, the upper segment of duodenum and jejunum can also be protected, thereby preventing or/and treating duodenal or/and jejunal inflammation or/and ulcers. The amount and period of superimposed administration is determined according to the time of gradual degradation or/and erosion or/and dissolution of the adhesive material in vivo.

A gastric-coated positioning immediate-release bioadhesive is provided, compared with the prior art (the invention patent “tissue conveyor used in sleeve gastrectomy and related using method”; the invention patent “releasable gastroplasty ring”; the invention patent “gastroplasty ring with single control”; Yang Kejun, Advantages of adjustable gastric band bariatric surgery. Shanghai Medicines, 2012, 33(8): 11; Yang Shen, et al., Clinical studies of intragastric balloon therapy for obesity. Chinese Medical Science, 2011, 1(6): 23-24; Mei Liwen, et al., Efficacy and safety evaluation of intragastric balloon therapy in patients with obesity. Chinese Medical Journal, 2007, 87(6): 388-391) and the like, by applying the positioning immediate-release bioadhesive, the user (patients with obesity, patients with diabetes and other people) does not need to go to the hospital, perform the operation or use endoscopy and further has no pain, the positioning immediate-release bioadhesive only needs to be orally taken, after the administration, the pH-sensitive coating material transfers the gastric-coated immediate-release bioadhesive (micro-particles or/and capsules or/and tablets) to the stomach in a positioning manner according to the differences in the intra-gastrointestinal pH value, and the gastric-coated immediate-release bioadhesive achieving the stomach rapidly or/and sharply degrades the coating material in a gastric pH environment. In the gastric cavity, the adhesive material in the gastric-coated immediate-release bioadhesive is rapidly, fully and completely released, disintegrated, floated, dissolved and swollen (the positioning immediate-release bioadhesive which is pressed into the tablets is also rapidly, fully and completely released, disintegrated, floated, dissolved and swollen due to the immediate-release disintegrant), further interacted with mucosal proteins or/and mucosal epithelial cells and the like to adhere thereto immediately after being in contact with gastric mucosa till the gastric-coated immediate-release bioadhesive is fully adhered to and covers the gastric mucosa or/and is embedded into folds and valley cracks of the mucosa; and the pylorus of the stomach further prolongs the retention time of the adhesive material in the stomach while reducing the reflux of the contents in the duodenum. Thus, the adhesive material is adhered to and covers on the gastric mucosa and the absorption of the stomach can be reduced. The amount and period of superimposed administration is determined according to the time of gradual degradation or/and erosion or/and dissolution of the adhesive material in vivo.

A gastric-coated or/and enteric-coated positioning immediate-release bioadhesive is provided, compared with the prior art (the invention patent “composition of hangover relief oral medicament and preparation process thereof”; the invention patent “anti-drunk and hangover relief composition and preparation method thereof”; the invention patent “oral absorption solid hangover relief effervescent preparation”) and the like, after the positioning immediate-release bioadhesive of the invention is orally taken, the pH-sensitive coating material transfers the gastric-coated or/and enteric-coated immediate-release bioadhesive (micro-particles or/and capsules or/and tablets) to the stomach or/and the duodenum and jejunum in a positioning manner according to the differences in the intra-gastrointestinal pH value, and the gastric-coated or/and enteric-coated immediate-release bioadhesive achieving the stomach or/and the duodenum and jejunum rapidly or/and sharply degrades the coating material in a corresponding pH environment. In the gastric cavity or/and the duodenum and jejunum cavity, the adhesive material in the gastric-coated or/and enteric-coated immediate-release bioadhesive is rapidly, fully and completely released, disintegrated, floated, dissolved and swollen (the positioning immediate-release bioadhesive which is pressed into the tablets is also rapidly, fully and completely released, disintegrated, floated, dissolved and swollen due to the immediate-release disintegrant), further interacted with mucosal proteins or/and mucosal epithelial cells and the like to adhere thereto immediately after being in contact with the mucosa of the stomach or/and the duodenum and jejunum till the gastric-coated or/and enteric-coated immediate-release bioadhesive is fully adhered to and covers the mucosa of the stomach or/and the duodenum and jejunum or/and is embedded into folds and valley cracks of the mucosa. Thus, the gastric-coated or/and enteric-coated immediate-release bioadhesive is just adhered to and covers the mucosa of the stomach or/and the duodenum and jejunum and not absorbed, thereby actively inhibiting the absorption of the wine by the stomach or/and the duodenum and jejunum; and as the gastric-coated or/and enteric-coated immediate-release bioadhesive covers the mucosa of the stomach or/and the duodenum and jejunum, the mucosa of the stomach or/and the duodenum and jejunum can also be protected, thereby preventing or/and treating inflammation or/and ulcers of the stomach or/and the duodenum and jejunum. The amount and period of superimposed administration is determined according to the time of gradual degradation or/and erosion or/and dissolution of the adhesive material in vivo.

### BRIEF DESCRIPTION OF THE DRAWINGS

FIG. 1 (positioning immediate-release bioadhesive tablet) and FIG. 2 (positioning immediate-release bioadhesive capsule) are structural schematic diagrams of the invention.

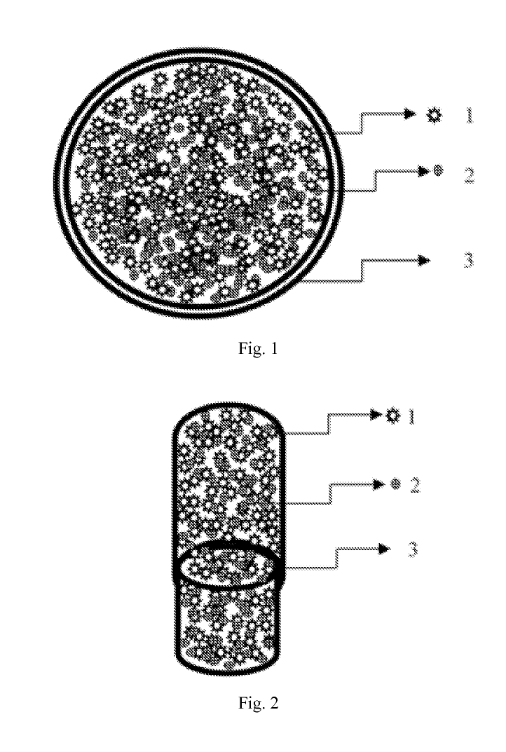

In FIG. 1 (positioning immediate-release bioadhesive tablet), components or parts represented by symbols are as follows: 1-bioadhesive micro-particles; 2-immediate-release disintegrant or/and diluent or/and lubricant or/and wetter and the like; and 3-gastric or enteric coating.

In FIG. 2 (positioning immediate-release bioadhesive capsule), components or parts represented by symbols are as follows: 1-bioadhesive micro-particles; 2-anti-sticking agent or/and diluent or/and lubricant or/and disintegrant and the like; and 3-gastric-coated or enteric-coated capsule shell.

### DETAILED DESCRIPTION OF THE EMBODIMENTS

The invention is further illustrated below in conjunction with the specific embodiments.

### Embodiment 1

The preparation of micro-particles is as follows: taking one part of solution formed by dissolving 10% of ethyl cellulose-carbomer 934P copolymer in anhydrous ethanol, stirring in a water bath at the temperature of 10° C. for 20 min, slowly dropping into 5 parts of liquid paraffin with 3% of span 85 at the temperature of 10° C. at a uniform speed, stirring for 30 min, performing decompression at 60° C. to remove ethanol, immediately placing into an ice bath to cool to form a solid, centrifugally separating the liquid paraffin, precipitating, washing with petroleum ether and drying in a drying box at 37° C. for 24 hours, screening by a 100-mesh screen rather than a 200-mesh screen; and observing the shape under an optical microscope.

### Embodiment 2

The gastric coating of the micro-particles can be as follows: placing the prepared micro-particles into a fluidized bed coating device for boiling and fluidizing, spraying a 6% ethanol hydroxypropyl methyl cellulose solution by the spray gun, blasting, drying, and exhausting and evaporating the solvent from an exhaust port to obtain the gastric-coated micro-particles with uniform coating thickness and no adhesion.

### Embodiment 3

The direct tabletting was performed as follows: uniformly mixing 1 part of carbomer 934P and 1 part of sodium carboxymethyl cellulose 2000 cp and directly tabletting powder till the thickness is 1 mm, the diameter is 3 mm and the hardness is about 4 kg/mm²; or performing wet process granulation and tabletting, wherein a binder can select a 70% ethanol solution with 5% of PVP_{K30}, a lubricant can select magnesium stearate (3%) and a filler can select pregelatinized starch.

### Embodiment 4

The tabletting of the micro-particles was performed as follows: screening raw materials and auxiliary materials by the 100-mesh screen, uniformly mixing, adding the binder, namely a 10% PVP water solution, to prepare a soft material, granulating, and drying at 60° C. for 1 h; and adding the magnesium stearate or/and the diluent or/and the wetter and the like, performing size stabilization and tabletting for preparation.

### Embodiment 5

The filling of gastric-coated capsules and the micro-particles can be performed as follows: using a high-efficiency coating machine, wherein the diameter of the spray nozzle is 1 mm, the atomization pressure is 0.1 MPa, the air volume is 60-80 m³/h, the material temperature is 23-25° C. and the spraying speed is 1.5-3.5 g/min; determining the thickness by a digital display micrometer, curing at 25° C. for 30-50 min, taking out the gastric-coated capsules after coating and drying at room temperature; filling the gastric-coated hollow capsules, sealing with a 10% ethyl cellulose solution and placing into a dryer for later use, wherein an appropriate amount of anti-sticking agent, namely magnesium stearate or silicon dioxide and the like, or the diluent, the lubricant, the disintegrant and the like can be added.

### Embodiment 6

The micro-particles were prepared by a drying method in a solution, comprising the following steps: taking 1.5 g of lactide-polyethylene glycol copolymer (PELA) with the weight ratio of lactide to polyethylene glycol of 90:10 and the molecular weight of the polyethylene glycol of 6000, dissolved in 20 ml of anhydrous ethanol, taking this as an internal phase; taking 100 ml of liquid paraffin with 2% of span 85 as an external phase; slowly dropping the internal phase into the external phase while magnetically stirring at a high speed, performing decompression at 60° C. to remove ethanol, immediately placing into an ice bath to cool to form a solid; centrifugally separating the liquid paraffin, precipitating, washing with petroleum ether and performing vacuum drying; and screening by a 100-mesh screen rather than a 200-mesh screen; and observing the shape under an optical microscope.

### Embodiment 7

The micro-particles were prepared by a single-agglomeration method, comprising the following steps: configuring type A gelatin with the molecular weight of 50000 into a 5% solution, adding an agglomerant, namely sodium sulfate while stirring at the temperature of 45° C., standing, separating, washing with cold isopropanol, then cross-linking and curing with an isopropanol solution with 10% of formaldehyde, dehydrating, and performing vacuum drying to obtain the micro-particles, wherein water can be used as a diluent; and repeatedly performing agglomeration and deagglomeration, observing the shape under the optical microscope till the formation of the appropriate shape and further cross-linking and curing.

### Embodiment 8

The micro-particles were prepared by a re-agglomeration method, comprising the following steps: adding water into a mixed solution with 10% of gelatin, 10% of gum arabic and 80% of water for gradual dilution, observing the shape under the optical microscope till the formation of an appropriate shape and further cross-linking and curing.

### Embodiment 9

The micro-particles were prepared by a temperature regulation method, comprising the following steps: dissolving a ternary system consisting of polyisobutylene, ethyl cellulose and cyclohexane at 80° C. to form a uniform solution, slowly cooling to 45° C. and further rapidly cooling to 25° C. to form the micro-particles.

### Embodiment 10

Gelatin nanospheres were prepared by a physical and chemical method, comprising the following steps: placing a 300 g/L gelatin solution into an equal amount of sesame oil for emulsification, placing an emulsion into the ice bath to gelatinize gelatin emulsion droplets, diluting with acetone, filtering with a 50 nm filter membrane, rinsing oil on nanospheres with the acetone, cross-linking with an acetone solution with 10% of formaldehyde for 10 min and drying for preparation.

### Embodiment 11

PLGA nano-particles were prepared by a precipitation method, comprising the following steps: ultrasonically dissolving 100 mg of PLGA in 6 ml of acetone, dropping into 40 ml of water solution with 0.03% of carbomer while magnetically stirring, stirring at room temperature at 500 rpm till an organic solvent evaporates completely, centrifugating at 4° C. and 15000 rpm for 30 min, discarding supernatant, removing a residual surfactant, re-dissolving a precipitate in Millipore water, washing with water for three times and drying to obtain the PLGA nano-particles.

### Embodiment 12

The micro-particles were prepared by an ion cross-linking method, comprising the following steps: dissolving chitosan in a dilute acetic acid water solution, swelling overnight, preparing into a 0.5% (w/v) chitosan solution, dissolving sodium tripolyphosphate in distilled water to prepare a 0.5% (w/v) solution, continuously performing magnetic stirring, and adding a sodium tripolyphosphate solution into the chitosan solution at the dropping speed of about 3 ml/min, wherein the solution gradually turns from clear to light blue opalescent, and the formation of the nano-particles is judged according to opalescence.

### Embodiment 13

The micro-particles were prepared by an electrostatic spraying method, comprising the following steps: dissolving the PLGA in trifluoroethanol at normal temperature for 48 hr, performing magnetic stirring till 15% w/v, transferring the solution into a micro-infusion pump connected with a high-pressure generator, regulating the voltage V to 5-35 kV, the receiving distance L to 9 cm and the flow rate f of the solution to 0.6 ml/h, performing electrospraying, receiving the obtained micro-particles by an aluminum foil receiving plate or a glass slide, and drying in a drying box for 2 d to obtain the nano-particles; and observing the appearance of the prepared micro-particles under a scanning electron microscope.

### Embodiment 14

The preparation of immediate-release tablets was as follows: screening sodium bicarbonate and magnesium hydroxide in the ratio of 1:2, 1% of magnesium stearate, 3% of cross-linked carboxymethylcellulose sodium and 10% of Starch 1500 by a 100-mesh screen, totally and uniformly mixing, and tabletting till the hardness is 6 kg/mm².

### Embodiment 15

The preparation of immediate-release tablets was as follows: screening 40% of mannitol, 35% of microcrystalline cellulose and an appropriate amount of lactose by a 100-mesh screen, performing equal-amount gradual increase and uniform mixing, adding a 5% polyvinylpyrrolidone K30 solution as the binder, granulating, drying at 60° C. for 1 h, performing size stabilization, and further uniformly mixing an appropriate amount of carboxymethylcellulose sodium and micro-powder silica gel and tabletting.

### Embodiment 16

Gastric coating was performed as follows: placing the immediate-release tablets into a coating pan, wherein the inclination angle of the coating pan was 45°, the air inlet temperature was 35±5° C., the atomization air pressure of a spray gun was 414 KPa, the spraying rate was 10 g/min, the temperature of the immediate-release tablets was controlled at 25±2° C., and the rotational speed was 15 r/min.

### Embodiment 17

The gastric coating is as follows: taking gastric-coated acrylic resin (No. VI) with a pH sensitive point of 1-2 and preparing into a 2.0% solution with acetone/ethanol (1/1, v/v), wherein the using amount of the additional agents is 10-20%; uniformly mixing and regulating the coating weight gain to 3%; regulating the rotational speed of a coating pan to enable tablet cores to roll, rotate and be polished in a parabolic manner for about 60±5 r/min; preheating the tablet cores by air which is blown in by an air blower, regulating the air inlet position and the air outlet speed at the temperature of about 50° C. to uniformly spray out a coating solution; observing the tablet cores after 15 min to find that the edges are smooth and have no defects or splits, the coated tablets can prevent sticking and coating films are uniform and smooth; taking out the tablet cores after coating, and drying in an oven at the temperature of about 60° C.; and weighing and taking the coating weight gain by percentage as a coating control index.

### Embodiment 18

The gastric coating of the micro-particles can be as follows: placing the prepared micro-particles into a fluidized bed coating device for boiling and fluidizing, spraying a 5-7% ethanol hydroxypropyl methyl cellulose solution by the spray gun, blasting, drying, and exhausting and evaporating the solvent from an exhaust port to obtain the gastric-coated micro-particles with uniform coating thickness and no adhesion.

### Embodiment 19

The filling of gastric-coated capsules and the micro-particles can be performed as follows: using a high-efficiency coating machine, wherein the diameter of the spray nozzle is 0.5-1.5 mm, the atomization pressure is 0.1 MPa, the air volume is 60-80 m³/h, the material temperature is 23-25° C. and the spraying speed is 1.5-2.5 g/min; determining the thickness by a digital display micrometer, curing at 25° C. for 30-40 min, taking out the gastric-coated capsules after coating and drying at room temperature; filling the gastric-coated hollow capsules, sealing with a 10% ethyl cellulose solution and placing into a dryer for later use, wherein an appropriate amount of anti-sticking agent, namely magnesium stearate or silicon dioxide and the like, or the diluent, the lubricant, the disintegrant and the like can be added.

### Embodiment 20

The disintegration determination of enteric-coated immediate-release tablets was as follows: by referring to a static method of the Center for Drug Evaluation, State Food and Drug Administration, placing a screen basket (with the hole inner diameter of 400 μm) into a test tube containing 2 ml of artificial intestinal fluid, further vertically placing into a water bath at 37° C., placing one enteric-coated immediate-release tablet into the screen basket after the temperature in the test tube rose, starting to count time from the moment that the enteric-coated immediate-release tablet was in contact with the artificial intestinal fluid till complete disintegration, then immediately lifting the screen basket away from the test tube, observing that there was no obvious residue on a screen mesh and testing 6 tablets in total, with the result of each tablet being less than 15 s.

### Embodiment 21

The disintegration determination of gastric-coated immediate-release tablets was as follows: by referring to a static method of the Center for Drug Evaluation, State Food and Drug Administration, placing a screen basket (with the hole inner diameter of 400 μm) into a test tube containing 2 ml of artificial gastric fluid, further vertically placing into a water bath at 37° C., placing one gastric-coated immediate-release tablet into the screen basket after the temperature in the test tube rose, starting to count time from the moment that the gastric-coated immediate-release tablet was in contact with the artificial gastric fluid till complete disintegration, then immediately lifting the screen basket away from the test tube, observing that there was no obvious residue on a screen mesh and testing 6 tablets in total, with the result of each tablet being less than 15 s.

### Embodiment 22

The in-vivo positioning test in rats was performed as follows: taking 30 SD rats with the body weight of 216.37±17.53 g, fasting for 5 h, performing intragastric administration with 200 enteric-coated immediate-release bioadhesive micro-particles and water, killing the rats immediately and at 10′ and 20′ after intragastric administration respectively, opening abdominal cavities of the rats, performing sharp dissection from the cardia to expose the gastric and intestinal cavity till the ileocecum in each rat, and observing the distribution of the enteric-coated immediate-release bioadhesive micro-particles in the gastrointestinal tract in each rat by naked eyes. The results showed that, in each rat, 160.70±17.33 integral and slightly swollen enteric-coated immediate-release bioadhesive micro-particles existed in the stomach and 35.90±15.47 swollen or dissolved enteric-coated immediate-release bioadhesive micro-particles immediately after intragastric administration; 1.40±1.96 integral and slightly swollen enteric-coated immediate-release bioadhesive micro-particles existed in the stomach and 186.40±7.76 swollen or dissolved enteric-coated immediate-release bioadhesive micro-particles existed in the duodenum at 10′ after intragastric administration; and 0.70±0.82 integral and slightly swollen enteric-coated immediate-release bioadhesive micro-particle existed in the stomach and 191.50±4.03 swollen or dissolved enteric-coated immediate-release bioadhesive micro-particles existed in the duodenum at 20′ after intragastric administration. It was thus clear that the enteric-coated immediate-release bioadhesive was dissolved in the duodenum in a positioning manner.

### Embodiment 23

The in-vivo positioning test in rats was performed as follows: taking 20 SD rats with the body weight of 223.17±20.04 g, fasting for 5 h, performing intragastric administration with 200 gastric-coated immediate-release bioadhesive micro-particles and water, killing the rats immediately and at 5′ and 15′ after intragastric administration respectively, opening abdominal cavities of the rats, performing sharp dissection from the cardia to expose the gastric and intestinal cavities in each rat, and observing the distribution of the gastric-coated immediate-release bioadhesive micro-particles in the gastrointestinal tract in each rat by naked eyes. The results showed that, 190.92±13.12 swollen or dissolved gastric-coated immediate-release bioadhesive micro-particles existed in the stomach at 5′ after intragastric administration and 193.75±7.84 swollen or dissolved gastric-coated immediate-release bioadhesive micro-particles existed in the stomach at 15′ after intragastric administration. It was thus clear that the gastric-coated immediate-release bioadhesive was dissolved in the stomach in a positioning manner.

### Embodiment 24

The acute toxicity test was performed as follows: taking 20 Kunming mice with the body weight of 22.75±2.63 g, randomly dividing the mice into two groups, performing ip (intraperitoneal injection) of a bioadhesive material leaching solution on the test group according to 50 ml/kg and performing ip of the equal amount of physiological saline on the control group; and observing general conditions, toxic reaction and number of dead animals at 24 h, 48 h and 72 h after injection respectively. The results showed that all the animals in the test group had no bradykinesia, weight loss, diarrhea, paralysis, respiratory depression, convulsion, death and other physical signs.

### Embodiment 25

The subacute toxicity test was performed as follows: taking 24 SD rats with the body weight of 214.61±18.72 g and randomly dividing the rats into two groups; preparing bioadhesive material fine powder into a 5% suspension with physiological saline, performing ig (intragastric administration) at 9 a.m. qod, and performing ig of the equal amount of physiological saline on the control group; and observing the general conditions and the body weight, killing 6 rats in each group at 2 W and 4 W respectively, taking the heart, liver, kidney and spleen tissues, weighing, fixing for pathological tissue sections, analyzing organ indexes (organ weight/animal weight) by SPSS12.0 statistical analysis software, adopting variance analysis between the groups, adopting t test in each group and taking p<0.05 as significant difference. The results showed that all the animals in the test group had no bradykinesia, weight loss and other physical signs, the organ indexes of the test group were as follows: heart: 0.454±0.062, liver: 3.203±0.254, kidney: 0.869±0.077 and spleen: 0.269±0.085, the organ indexes of the control group were as follows: heart: 0.463±0.039, liver: 3.317±0.472, kidney: 0.878±0.071 and spleen: 0.273±0.064, and compared with the control group, the differences in the heart, liver, kidney, spleen and other organs had no significance (P>0.05). No obvious abnormalities were found in the pathological tissue sections.

### Embodiment 26

The skin irritation test was performed as follows: taking 3 New Zealand rabbits with the body weight of 2.75±0.13 kg, adding 10 g of sterile bioadhesive material fine powder into 50 ml of physiological saline, performing high-temperature and high-pressure sterilization, leaching at 37° C. for 72 h, centrifugating at 2500 rpm for 5 min and taking supernatant; performing skin preparation with an area of about 10×10 cm on the two sides of the back of each rabbit respectively, performing id (intradermal injection) on 10 sites on one side with 0.5 ml of leaching solution, performing id of the equal amount of physiological saline on the other side, and observing the physical signs of the sites at 1 h, 24 h, 48 h and 72 h after injection. The results showed that there were no obvious physical signs of redness and swelling, fester, exudate and the like on the test side and the control side of each rabbit at 1 h, 24 h, 48 h and 72 h after injection and no obvious skin irritation signs appeared.

### Embodiment 27

The in-vitro gastric mucosal adhesion test was performed as follows: taking 8 Kunming mice with the body weight of 21.36±2.41 g, fasting for 24 h (but supplying water), killing the mice by cervical dislocation, immediately taking the stomach of each mouse, cutting open along the greater curvature of the stomach from the cardia to the pylorus, spreading on a glass slide for each mouse, evenly scattering gastric-coated positioning immediate-release bioadhesive micro-particles, placing into a container containing a saturated sodium chloride solution, closing, keeping humidity for 10 min, taking out, rinsing for 5 min with a hydrochloride and sodium chloride solution with the pH of 1.3 at 20 ml/min, observing the shedding area of the micro-particles, performing equidistant digital photographing and performing image analysis if necessary to compare the shedding area. The results showed that, according to the observation by the naked eyes, no obvious shedding of the gastric-coated positioning immediate-release bioadhesive micro-particles occurred.

### Embodiment 28

The in-vitro gastric mucosal adhesion test was performed as follows: taking 10 SD rats with the body weight of 227.83±19.41 g, fasting for 24 h (but supplying water), taking the stomach as above, pressing the gastric-coated immediate-release bioadhesive into tablets, wetting with artificial gastric fluid for 10 min, then bridging with a torsion balance, fixing and zeroing a pointer of the balance; placing a culture dish (while keeping humidity) storing gastric mucosa on a lifting platform, regulating the lifting platform to enable the gastric mucosa to be just in contact with and adhere to the gastric-coated immediate-release bioadhesive after wetting, applying the pulling force of 2 mg/s to the gastric-coated immediate-release bioadhesive after 10 min till the mucosa was just separated from the gastric-coated immediate-release bioadhesive and recording the reading of the balance. The results showed that the gastric-coated immediate-release bioadhesive had a good adhesion effect on the gastric mucosa.

### Embodiment 29

The in-vitro intestinal mucosal adhesion test was performed as follows: taking 10 SD rats with the body weight of 231.42±15.89 g, fasting for 24 h (but supplying water), killing the rats by cervical dislocation, immediately taking the upper segment from duodenum to duodenum of each rat, spreading, rinsing with a phosphate buffered saline with the pH of 6.8, placing into a container containing a saturated sodium chloride solution, closing and keeping humidity; pressing the enteric-coated immediate-release bioadhesive into tablets, wetting with the phosphate buffered saline with the pH of 6.8 for 10 min, bridging with a torsion balance, fixing and zeroing a pointer of the balance; placing a culture dish (while keeping humidity) storing small intestinal mucosa on a lifting platform, regulating the lifting platform to enable the small intestinal mucosa to be just in contact with and adhere to the enteric-coated immediate-release bioadhesive after wetting, applying the pulling force of 2 mg/s to the enteric-coated immediate-release bioadhesive after 10 min till the mucosa was just separated from the enteric-coated immediate-release bioadhesive and recording the reading of the balance. The results showed that the enteric-coated immediate-release bioadhesive had a good adhesion effect on the mucosa at the upper segment from the duodenum to the duodenum.

### Embodiment 30

The in-vivo perfusion mucosal adhesion test (enteric-coated) was performed as follows: taking 6 SD rats with the body weight of 253.10±19.24 g, fasting for 24 h (but supplying water), anesthetizing with urethane, cutting open each rat along the midline of abdomen, ligating the cardia, performing blunt dissection on the whole intestinal segment including the stomach and small intestines, rinsing the contents, performing distal ligation, respectively connecting the proximal end of the stomach and the distal end of the small intestines with glass tubes, and connecting the glass tube at the proximal end of the stomach with a peristaltic pump; taking 200 enteric-coated immediate-release bioadhesive micro-particles, suspending in 100 ml of physiological saline, perfusing a suspension of the enteric-coated immediate-release bioadhesive micro-particles, collecting effluent, counting the number of the enteric-coated immediate-release bioadhesive micro-particles in the effluent and calculating the retention rate of the coating micro-particles at different parts. The adhesion performance of the enteric-coated immediate-release bioadhesive micro-particles was different at different parts, and the adhesion performance in the stomach and the small intestines was 3.53±0.21% and 87.36±5.59% respectively.

### Embodiment 31

The in-vivo perfusion mucosal adhesion test (gastric-coated) was performed as follows: taking 6 SD rats with the body weight of 244.31±17.37 g, fasting for 24 h (but supplying water), anesthetizing with urethane, cutting open each rat along the midline of abdomen, ligating the cardia, performing blunt dissection on the whole intestinal segment including the stomach and small intestines, rinsing the contents, performing distal ligation, respectively connecting the proximal end of the stomach and the distal end of the small intestines with glass tubes, and connecting the glass tube at the proximal end of the stomach with a peristaltic pump; taking 200 gastric-coated immediate-release bioadhesive micro-particles, suspending in 100 ml of physiological saline, perfusing a suspension of the gastric-coated immediate-release bioadhesive micro-particles, collecting effluent, counting the number of the gastric-coated immediate-release bioadhesive micro-particles in the effluent and calculating the retention rate of the coating micro-particles at different parts. The adhesion performance of the gastric-coated immediate-release bioadhesive micro-particles was different at different parts, and the adhesion performance in the stomach and the small intestines was 90.13±3.74% and 8.45±0.67% respectively.

### Embodiment 32

The in-vitro perfusion mucosal adhesion test (enteric-coated) was performed as follows: taking 10 SD rats with the body weight of 230.07±15.83 g, killing the rats by cervical dislocation, cutting open each rat along the midline of abdomen, taking out the duodenum, rinsing the contents with the phosphate buffered saline with the pH of 6.8, attaching into an inclined fixed tube, dropping a suspension of the micro-particles into the inclined tube from an upper opening, recording the number the micro-particles which were eluted out at a lower opening, and calculating the retention rate of the micro-particles according to the formula of the retention rate to get the result that the retention rate of the micro-particles was 85.15±7.46%.

### Embodiment 33

The determination of the adhesion performance on porcine small intestines was performed as follows: rinsing Bama porcine small intestines with the phosphate buffered saline, fixing the apical side in a culture dish, fixing the culture dish on an electronic balance, wetting a sufficient amount of enteric-coated adhesive micro-particles with the phosphate buffered saline for 2 min, being in contact with the mucosa of the small intestines under the pressure of 5 g of a pressure panel for 5 min, slowly regulating the pressure panel at uniform speed, removing the pressure, separating, recording the reading of the balance when the enteric-coated adhesive micro-particles were just separated from the mucosa, converting the recorded grams to the unit of Newtons and further dividing by the adhesion area to obtain the adhesion force. The result showed that the enteric-coated adhesive micro-particles had a good adhesion effect on the mucosa.

### Embodiment 34

The test for preventing and treating alcoholism was performed as follows: taking 20 Kunming mice with the body weight of 23.47±2.11 g, fasting for 12 h and randomly dividing the mice into two groups, namely a bioadhesive group and a control group; firstly performing intragastric administration on the bioadhesive group with the enteric-coated immediate-release bioadhesive according to 20 g/kg body weight, then performing intragastric administration with the gastric-coated immediate-release bioadhesive according to 20 g/kg body weight, performing intragastric administration on the control group with the equal volume of physiological saline, performing intragastric administration on each group with Erguotou liquor having the alcohol content of 56% (v/v) according to 10 ml/kg body weight after 30 min and recording the loss of righting reflex and time of each mouse (after liquoring, each mouse was put into a supine state, if the state was kept for over 30 s, the loss of righting reflex occurred, namely the mouse was drunk, and otherwise, the mouse was not drunk). The results showed that 7 mice in the bioadhesive group were not drunk and 10 mice in the control group were all drunk.

### Embodiment 35

The test for preventing and treating gastric and intestinal mucosal inflammation or/and ulcers was performed as follows: taking 40 Kunming mice with the body weight of 25.13±2.79 g, randomly dividing the mice into four groups (namely a control group A, a control group B, a pre-bioadhesive group and a post-bioadhesive group), fasting for 12 h, firstly performing intragastric administration on the pre-bioadhesive group with the enteric-coated immediate-release bioadhesive according to 20 g/kg body weight, then performing intragastric administration with the gastric-coated immediate-release bioadhesive according to 20 g/kg body weight, and performing intragastric administration on the control group A with the equal volume of physiological saline); performing intragastric administration on each group with Erguotou liquor having the alcohol content of 56% (v/v) according to 15 ml/kg body weight; after 60 min, firstly performing intragastric administration on the post-bioadhesive group with the enteric-coated immediate-release bioadhesive according to 20 g/kg body weight, further performing intragastric administration with the gastric-coated immediate-release bioadhesive according to 20 g/kg body weight and performing intragastric administration on the control group B with the equal volume of physiological saline; after 5 h, killing the mice by cervical dislocation, cutting open each mouse along the midline of abdomen, taking out the stomach and the duodenum, cutting open along the greater curvature of the stomach, rinsing with the physiological saline, sucking dry with filter paper, observing mucosal injuries by the naked eyes, cutting off and taking gastric mucosa and duodenal mucosa, fixing with 3.7% polyoxymethylene, embedding with conventional paraffin, slicing, performing HE staining and observing tissue pathological changes of the gastric mucosa and the duodenal mucosa under an optical microscope. The results showed that, according to the observation by the naked eyes, the gastric mucosa and the duodenal mucosa in the pre-bioadhesive group were covered with the bioadhesive thin layers and had no obvious injuries, the gastric mucosa and the duodenal mucosa in the post-bioadhesive group were covered with the bioadhesive thin layer and had visible mild injuries, the gastric mucosa and the duodenal mucosa in the control group A and the control group B had obviously visible injuries, and such situation was more severe in the control group B; and it could be seen from the pathological sections that the gastric mucosa and the duodenal mucosa in the control group A and the control group B widely had hyperemia and edema, as well as inflammatory cell infiltration and mainly contained neutrophiles, epithelial cells fell off due to necrosis, the mucosa in the control group B had erosion, ulcers and more bleeding and necrosis, the gastric mucosa and the duodenal mucosa in the pre-bioadhesive group had integral tissue structures, glands were neatly arranged and clear in layers, and the edema and the inflammatory cell infiltration could be seen in the lower layers of the gastric mucosa and the duodenal mucosa in the post-bioadhesive group.

### Embodiment 36

The test for preventing and treating obesity was performed as follows: taking 20 male SD rats ablactated at 21 days, with the body weight of 54.77±6.13 g, randomly dividing the rats into two groups (a control group and a bioadhesive group), feeding the two groups with high-fat and high-nutrition feed for 3 weeks, and during the period, firstly performing intragastric administration on the bioadhesive group with the enteric-coated immediate-release bioadhesive according to 20 g/kg body weight, further performing intragastric administration with the gastric-coated immediate-release bioadhesive according to 20 g/kg body weight bid and performing intragastric administration on the control group with the equal volume of physiological saline; and analyzing by SPSS12.0 statistical analysis software, adopting variance analysis between the groups, adopting t test in each group and taking p<0.05 as significant difference. After feeding with the high-fat and high-nutrition feed for 3 weeks, obvious obesity occurred in the control group (136.25±15.08 g), no obvious obesity occurred in the bioadhesive group (109.84±12.23 g) and the difference between the two groups had great significance (P<0.01).

### Embodiment 37

The test for preventing and treating obesity was performed as follows: taking 20 male SD rats ablactated at 21 days, with the body weight of 52.96±5.87 g, randomly dividing the rats into two groups (a control group and a bioadhesive group), feeding the two groups with high-fat and high-nutrition feed in the first 3 weeks, and feeding the two groups with ordinary feed in the next three weeks; after the next three weeks, firstly performing intragastric administration on the bioadhesive group with the enteric-coated immediate-release bioadhesive according to 20 g/kg body weight, further performing intragastric administration with the gastric-coated immediate-release bioadhesive according to 20 g/kg body weight bid and performing intragastric administration on the control group with the equal volume of physiological saline; and analyzing by SPSS12.0 statistical analysis software, adopting variance analysis between the groups, adopting t test in each group and taking p<0.05 as significant difference. The results showed that the body weight in the control group was 286.13±19.45 g, the body weight in the bioadhesive group was 247.23±25.76 g and the difference between the two groups had great significance (P<0.01).

### Embodiment 38

The test for preventing and treating diabetes was performed as follows: taking 30 male SD mats with the body weight of 224.14±9.92 g, feeding for one week, observing the body weight, blood sugar and other physiological indexes of each rat and enabling the rats to adapt to a new environment so as to be conductive to modeling; starting to model after 1 week and fasting for 6 h; preparing STZ with a citric acid buffered solution under dark and ice bath conditions, performing ip according to 50 mg/kg and smearing a little chlortetracycline ointment at the injection part of each rat after injection, wherein each rat could drink water immediately after injection and started to take food after 4 h; measuring blood sugar after 72 h, wherein the rat with the blood sugar value of ≧16.7 mM/L was determined to be modeled successfully; randomly taking 20 SD rats which were modeled successfully, randomly dividing the rats into two groups (namely a control group and a bioadhesive group), firstly performing intragastric administration on the bioadhesive group with the enteric-coated immediate-release bioadhesive according to 20 g/kg body weight, further performing intragastric administration with the gastric-coated immediate-release bioadhesive according to 20 g/kg bid, and performing intragastric administration on the control group with the equal volume of physiological saline); and analyzing by SPSS12.0 statistical analysis software, adopting variance analysis between the groups, adopting t test in each group and taking p<0.05 as significant difference. After 6 weeks, the blood sugar value of the bioadhesive group was 9.43±3.75 mM/L, the blood sugar value of the control group was 25.71±5.93 mM/L and the difference between the two groups had great significance (P<0.01).

The parts which are not involved in the invention contain the same prior art or can be implemented by adopting the prior art.

1. A positioning immediate-release bioadhesive, wherein the positioning immediate-release bioadhesive is prepared by preparing a biocompatible bioadhesive material into micro-particles, externally adding an immediate-release disintegrant, tabletting and then performing enteric coating; or performing enteric coating on the micro-particles; or filling into enteric-coated hollow capsules; or directly tabletting the bioadhesive material, the immediate-release disintegrant and other additional agents and then performing enteric coating; and the positioning immediate-release bioadhesive is capable of adhering to and cover mucosa of the upper segment of duodenum and jejunum after administration and is capable of preventing or/and treat diabetes and obesity, weaken alcohol absorption and prevent or/and treat duodenal inflammation or/and ulcers.

2. A positioning immediate-release bioadhesive, wherein the positioning immediate-release bioadhesive is prepared by preparing a biocompatible bioadhesive material into micro-particles, externally adding an immediate-release disintegrant, tabletting and then performing gastric coating; or performing gastric coating on the micro-particles; or filling into gastric-coated hollow capsules; or directly tabletting the bioadhesive material, the immediate-release disintegrant and other additional agents and then performing gastric coating; and the positioning immediate-release bioadhesive is capable of adhering to and cover gastric mucosa, weaken alcohol absorption in stomach, prevent or/and treat obesity and prevent or/and treat gastritis or/and ulcers.

3. The positioning immediate-release bioadhesive according to claim 1, wherein the biocompatible bioadhesive material comprises, but is not limited to, carbomer (CP), hydroxypropyl methylcellulose (HPMC), hydroxypropyl cellulose (HPC), ethylenediamine-modified polylactic acid (EMPLA), polytetrafluoroethylene, polylactic acid-glycolic acid (PLGA), polylactic acid-caprolactone (PCL-b-LA), poly-ε-caprolactone (PCL), silicone oil, silicone rubber, polyester-polyether copolymer, grafted polylactic acid, gelatin, bletilla hyacinthine gum, alginate, cellulose derivatives, chitosan, lectin (phytohaemagglutinin), tomato lectin, N-(2-hydroxypropyl) methacrylamide copolymer and other materials.

4. The positioning immediate-release bioadhesive according to claim 1, wherein the immediate-release disintegrant comprises, but is not limited to, polyvinylpyrrolidone, carboxymethylcellulose sodium (CMC-Na), carboxymethylcellulose calcium, carboxymethyl starch sodium (CMS-Na), microcrystalline cellulose, low-substituted hydroxypropyl cellulose, magnesium stearate, alginate, pregelatinized starch, dextran and other materials and cross-linked matters thereof.

5. The positioning immediate-release bioadhesive according to claim 1, the preparation of the micro-particles comprises, but is not limited to a solvent evaporation method, a spray drying method, a phase separation method, an electrospraying method, an acoustic excitation atomization method and other methods; and the preparation steps are as follows: a. preparing the biocompatible bioadhesive material into the micro-particles, externally adding the immediate-release disintegrant and other additional agents, tabletting and then performing enteric coating; b. preparing the biocompatible bioadhesive material into the micro-particles and directly performing enteric coating on the micro-particles; c. preparing the biocompatible bioadhesive material into the micro-particles and filling the micro-particles and the additional agents into the enteric-coated hollow capsules together; and d. preparing the biocompatible bioadhesive material into the micro-particles, directly tabletting the micro-particles, the immediate-release disintegrant and other additional agents together and performing enteric coating.

6. The positioning immediate-release bioadhesive according to claim 2, wherein the biocompatible bioadhesive material comprises, but is not limited to, carbomer (CP), hydroxypropyl methylcellulose (HPMC), hydroxypropyl cellulose (HPC), ethylenediamine-modified polylactic acid (EMPLA), polytetrafluoroethylene, polylactic acid-glycolic acid (PLGA), polylactic acid-caprolactone (PCL-b-LA), poly-ε-caprolactone (PCL), silicone oil, silicone rubber, polyester-polyether copolymer, grafted polylactic acid, gelatin, bletilla hyacinthine gum, alginate, cellulose derivatives, chitosan, lectin (phytohaemagglutinin), tomato lectin, N-(2-hydroxypropyl) methacrylamide copolymer and other materials.

7. The positioning immediate-release bioadhesive according to claim 2, wherein a gastric coating material comprises, but is not limited to hydroxypropyl methylcellulose (HPMC), methyl cellulose (MC), polyvinyl alcohol (PVA), hydroxypropyl cellulose (HPC), polyethylene glycol (PEG), polyvinyl acetal diethylamino acetate (AEA), Eudragit E type, gastric-coated acrylic resin and other materials.

8. The positioning immediate-release bioadhesive according to claim 2, wherein the preparation of the micro-particles comprises, but is not limited to a solvent evaporation method, a spray drying method, a phase separation method, an electrospraying method, an acoustic excitation atomization method and other methods; and the preparation steps are as follows: a) preparing the biocompatible bioadhesive material into the micro-particles, externally adding the immediate-release disintegrant and other additional agents, tabletting and then performing gastric coating; b) preparing the biocompatible bioadhesive material into the micro-particles and directly performing gastric coating on the micro-particles; c) preparing the biocompatible bioadhesive material into the micro-particles and filling the micro-particles and the additional agents into the gastric-coated hollow capsules together; and d) preparing the biocompatible bioadhesive material into the micro-particles, directly tabletting the micro-particles, the immediate-release disintegrant and other additional agents together and performing gastric coating.

9. The positioning immediate-release bioadhesive according to claim 1, wherein the positioning immediate-release bioadhesive is prepared into a medical device for preventing or/and treating diabetes, obesity, duodenal inflammation or/and ulcers and weakening alcohol absorption.

10. The positioning immediate-release bioadhesive according to claim 2, wherein the positioning immediate-release bioadhesive is prepared into a medical device for preventing or/and treating obesity, gastritis or/and ulcers and weakening alcohol absorption.

11. The positioning immediate-release bioadhesive according to claim 1, wherein an enteric coating material comprises, but is not limited to, Eudragit L type, Eudragit S type, cellulose acetate phthalate (CAP), 1,2,4-cellulose acetate trimellitate (CAT), cellulose acetate succinate (CAS), hydroxypropylmethylcellulose phthalate (HPMCP), 1,2,4-hydroxypropylmethylcellulose trimellitate (HPMCT), hydroxypropylmethylcellulose acetate succinate (HPMCAS), PAVHB, calcium alginate, ethanol-grafted styrene maleic anhydride copolymer, chitosan, sodium alginate, pH-sensitive hydrogel polymethacrylic acid (PMAA), guar gum/polyacrylic acid (GG/PAA), acrylic acid and acrylamide copolymerized and grafted hemicellulose hydrogel, carboxymethyl chitosan hydrogel (CMCSG), methacrylate polymer, ethyl cellulose, opadry, acrylic resin No. II, No. III and No. IV and other materials.

12. The positioning immediate-release bioadhesive according to claim 2, wherein the immediate-release disintegrant comprises, but is not limited to, polyvinylpyrrolidone, carboxymethylcellulose sodium (CMC-Na), carboxymethylcellulose calcium, carboxymethyl starch sodium (CMS-Na), microcrystalline cellulose, low-substituted hydroxypropyl cellulose, magnesium stearate, alginate, pregelatinized starch, dextran and other materials and cross-linked matters thereof.
